(19) United States
(12) Patent Application Publication
(10) Pub. No.: US 2025/0177622 A1
(43) Pub. Date: Jun. 5, 2025

(54) IT ARCHITECTURE FOR BLOOD TREATMENT SYSTEMS

(71) Applicant: VIVONIC GMBH, Sailauf (DE)

(72) Inventor: Allen BASSALY, Bad Homburg (DE)

(73) Assignee: VIVONIC GMBH, Sailauf (DE)

(21) Appl. No.: 18/714,100

(22) PCT Filed: Dec. 15, 2022
(86) PCT No.: PCT/EP2022/086077

§ 371 Date: May 29, 2024

(30) Foreign Application Priority Data
Dec. 15, 2021 (DE) 10 2021 133 253.2

(51) Int. Cl. A61M 1/16; G16H 20/17; G16H 40/63

(52) U.S. Cl. CPC A61M 1/1601; A61M 1/1656; G16H 20/17; G16H 40/63; A61M 2205/17; A61M 2205/3584; A61M 2205/502

(57) ABSTRACT

The present invention relates to a device for preparing a medical fluid and/or for monitoring the preparation of a medical fluid, having a housing, a first control unit that controls the device in operation, and an access control unit that forms at least one interface between the device and the outside world, wherein the first control unit is configured to also control the device on a failure of the access control unit, and the control unit and the access control unit are each configured on a separate circuit board arranged in the housing and the access control unit furthermore has a communication unit for communication with the outside world and has a safety unit that is formed separately from the communication unit and that is configured to check every communication between the first control unit and the outside world over the interfaces and only to permit it after a determined technical safety assessment.

The present invention relates to an IT architecture for devices that are typically used in the context of blood treatment systems. Such devices are, for example, blood treatment devices such as dialysis machines, a device for water preparation, preferably for preparing dialysis water such as reverse osmosis systems, water pre-treatment devices disposed upstream of a reverse osmosis system, or filtration systems, or devices for preparing a medical fluid such as concentrate preparation systems, for example for dialyzate or dialyzate concentrate.

Devices used in the medical sector are, as is known, subject to a complex licensing process that usually has to be repeated when a corresponding device is to be adapted or modified. A relatively slight change to a medical device such as a change in the design of the user interface (GU!), for example, already makes it necessary to license the total medical device again.

This has the result that the optical appearance: of the user interfaces of medical devices can typically only be adapted with great difficulty or not at all.

The underlying object of the present invention against this background is to alleviate or even to fully eliminate the problems of the prior art. It is in particular the underlying object of the present invention to provide a flexible and inexpensive possibility of adapting or updating the software and/or the optical appearance: of a user interface and simultaneously to ensure consistently high patient safety.

This object is achieved by the subject matters of the independent claims. Advantageous further developments of the invention form the subject matter of the dependent claims.

A device for preparing a medical fluid and/or for monitoring the preparation of a medical fluid is accordingly provided comprising: a housing, a first control unit that controls the device in operation, and an access control unit that forms interfaces between the device and the outside world, wherein the first control unit is configured to also control the device on a failure of the access control unit, and the control unit and the access control unit are each configured on a separate circuit board arranged in the housing and the access control unit furthermore has a communication unit for communication with the outside world and has a safety unit that is formed separately from the communication unit and that is configured to check every communication of the first control unit over the interfaces and only to permit it after a determined technical safety assessment.

The control unit is therefore so-to-say preferably configured also to autonomously control the device on a failure of the access control unit. In this context, autonomous means that the control unit can control the device on any disruption of the communication with the outside world or of the operation of the access control unit up to the failure of the access control unit without limitation and independently thereof.

An advantage can here comprise a device in accordance with the invention being able to be operated without any limitation of cyber safety or of the connectivity possibilities without any external gateway. It can in other words be considered such that gateway functionality is integrated in the device in a manner adapted to the environment of the device.

In general, a device in accordance with the invention can also change into emergency operation on any disruption of the communication with the outside world or of the operation of the access control unit up to the failure of the access control unit and can optionally preferably use a further control unit for this purpose that is provided for emergency operation.

In accordance with an embodiment of the invention, the safety unit is in communication with the outside world over at least two separate communication paths, with a first communication path coming from the outside first leading to the communication unit and thereupon to the safety unit and a second communication path coming from the outside leading directly to the safety unit and the safety unit being configured to check any communication of the first control unit over the first and second communication paths and only to permit it after a determined technical safety assessment.

The communication unit and the safety unit are preferably implemented as two separate web servers or as two separate and mutually autonomous parts of a communication module, for example of a web server. The communication unit and the safety unit are further preferably implemented as two separate and mutually autonomous parts of a so-called communication module implemented as software. The functional extent of web servers almost has the total functional extent of communication modules. The design as a communication module is therefore to be preferred over web servers, on the one hand, but, on the other hand, it appears reasonable to design web servers as a part or as parts of such a communication module that cover a large part of the functionality.

The cyber safety of the device can be increased in this manner.

A device in accordance with the invention of a second control unit can furthermore be equipped that controls the device in emergency operation, with the second control unit being configured on a separate, third circuit board arranged in the housing and the safety unit furthermore being configured to check every communication of the second control unit with the outside world.

The safety unit preferably forms a technical safety separation between a first region of the device and a second region of the device, with the first region having the communication unit and the second region having the first, and optionally the second, control unit.

The components of the device relevant to a licensing as a medical device can in this manner be separated or screened from other components of the device, such as the communication unit by means of the safety unit. This can advantageously make it possible that e.g. the communication unit can be changed by updates while the components licensed as a medical device continue to be unchanged and their licensing is therefore not questioned. Or a licensing or a re-licensing is facilitated by this separation.

The communication unit is preferably configured to provide a communication path for network communication, for example in that it hosts a web site by means of a web server and/or hosts a cloud service and/or provides a connection to a cloud server.

Since the communication unit is separated by means of the safety unit from the control units that represent components of the device relevant to a licensing as a medical device, a communication of the device with the outside world can take place without the licensing of the device as a medical device being affected thereby. Or the licensing or at least the re-licensing as a medical device—after a change to the components disposed outside the safety unit—can thereby be substantially simplified because the changes in the area of the communication unit (e.g. updating of the communication protocols or of other communication means to increase cyber security) are restricted to an area of the device that is outside the technical safety separation and thus does not affect the area classified as a medical device, for example.

At least information from the first control unit can preferably be transferred to the outside by means of the first communication path and information from the first control unit can be transferred to the outside by means of the second communication path and/or information can be transferred from the outside to the first control unit and/or access to the first safety unit can take place from the outside if the safety unit has determined a technical safety assessment. In this respect, the second communication path leads directly from the outside to the first control unit to the extent that it only leads via the safety unit and not via the communication unit. Control access can take place over this communication via the direct transfer of commands to the control unit from the outside.

A device in accordance with the invention furthermore preferably has at least one display unit that is part of the device and is preferably designed as a screen, with the display unit being connected to the first and/or second control unit(s). This display unit is preferably designed, for example, as a screen or touchscreen and installed in or at the device.

Alternatively or additionally, a device in accordance with the invention can have a further display unit that is connected or connectable to the communication unit, with the communication unit preferably being configured to determine and/or to adapt contents displayed by the display unit. This display unit can, for example, be designed as an external end device such as a tablet or as a laptop associated with the device.

Inputs, preferably with respect to the operation of the device are preferably still able to be made by means of the display unit(s). A direct and local control of the device can, for example, take place by means of the display unit, e.g. in that control commands are input manually. The display units can have input means such as a keyboard or switches, buttons, etc. or can sense a touching by the user (e.g. touchscreens) for the purpose of inputs.

Websites can, for example, be adapted by means of the communication unit that are invokable by means of the display unit so that the appearance presented to the user on the display unit can be adapted or updated more flexibly.

A device in accordance with the invention can, for example, be a medical device and/or a device for providing medical water or medical solutions. A device in accordance with the invention can, however, also only monitor the medical device and/or a device for providing mechanical water or medical solutions.

It is equally conceivable that the device is a blood treatment device, in particular a dialysis machine.

The device can also be a device for water pre-treatment including different treatment stages such as physical filters, active carbon filters, softening agents, a reverse osmosis system or other pure water system for providing dialysis water, a concentrate mixing system for dialyzate concentrate, a technical water control center, a mixing system for the central preparation of dialyzate, or another device that is used in the preparation of liquid media for dialysis.

The device is preferably further configured to detect a parameter of a medical fluid by means of at least one sensor, with the sensor either being a component of the device or being connected to the device via a line, for example an electrical line, a wireless connection, or a fluid line. The device in accordance with the invention can preferably monitor the provision of a liquid medium for blood treatment, of medical water, or of another medical solution by means of the sensor.

The present invention further relates to a use of a device in accordance with the invention in a clinic or in a dialysis center and/or for preparing a medical fluid and/or for water preparation, preferably for preparing a liquid medium for dialysis such as dialysis water, dialyzate concentrate, or dialyzate.

Expressed in other words, the invention can be described as follows:

A device is accordingly provided having a housing, a first control unit that controls the device in normal operation, and an access control unit that forms an interface between the device and the outside world, wherein the control unit and the access control unit are each configured on a separate circuit board arranged in the housing, and the access control unit furthermore has a communication unit by means of which aspects of the device can be adapted by a user and a safety unit that is formed separately from the communication unit and that is configured to check every communication of the first control unit with the outside world.

The access control unit is interposed in the communication between the outside world and the control unit(s) of the device. While the control unit(s) directly controls/control the operation of the device and is/are thus a part of the medical device and thus relevant to the licensing in a case e.g. in which the device is a blood treatment device, the access control unit only forwards information such as control commands from the outside world to the control unit(s) of the device without itself carrying out a control.

The access control unit is thus not necessarily deemed to be a part of the medical device with respect to a licensing process as a medical device so that changes of the access control unit do not require any new licensing of the device.

The safety unit here preferably forms a technical safety separation between a first region of the device and a second region of the device, with the first region having the communication unit and the second region having the first, and/or the second, control units.

In other words, two separate regions are provided by the safety unit and the functions of the control of the medical device and of the adaptation or updating, e.g. of the optical appearance of the user interface, and the communication with the outside world required for this purpose are clearly separated from one another.

it is thus possible to carry out an updating of the user interface without the components, in particular the control units, relevant to the control or the operation and thus to the licensing process of the medical device thereby being affected in any manner.

The components whose change would make a new licensing of the medical device necessary, in particular the control units, are thus screened by the safety unit and decoupled from the communication unit. Two independent regions in which the functions of the medical device relevant to the licensing are present separately from other components, for example the communication unit or also a communication client for data exchange with the outside world, are thus created by the separating effect of the safety unit.

A particular advantage here comprises both the access control unit and the control unit(s) being provided within the device, in particular within a housing of the device, and preferably each being present on separate circuit boards.

The necessity of separate devices is thus dispensed with since the required IT infrastructure is already installed in every device in accordance with the invention. The strict, also spatial, separation of the access control unit and of the control unit(s) within the device improves patient safety since a particularly reliable separation of the regions can thus be ensured.

The communication unit and the safety unit are preferably implemented as two separate web servers or as two separate parts of a web server that are present on the same circuit board as the access control unit. Different embodiments than an embodiment as a web server would also be possible as long as a clear separation of the communication unit and the safety unit is ensured.

The communication unit preferably generates a user interface on a display unit of the device. The communication unit preferably has no direct access to or no direct communication with the control unit(s) of the device, but can only communicate therewith via the safety unit.

In other words, the safety unit preferably acts as a kind of firewall in the communication with the control unit(s) of the device and, for example, carries out functions of authentication, data packet verification, audit protocolling, and preparation and checking of user models. Only signals positively checked by the safety unit with respect to the technical safety assessment are forwarded from the safety unit to the control unit(s).

A device in accordance with the invention can generally also be operated autonomously by the first and/or second control units on a failure of the access control unit.

To be ideally equipped for emergency operation, a device in accordance with the invention can furthermore have a second control unit that is configured to control the device in emergency operation, for example when there is no connection to the outside world and thus no possibility of remote access.

The second control unit is preferably configured on a separate circuit board arranged in the housing. The second control unit is also screened to the outside by the safety unit in a similar manner to the first control unit.

In other words, the safety unit is thus preferably still configured to check every communication of the second control unit with the outside world. The communication of the second control unit with the outside world thus also preferably does not run directly, but rather indirectly via the control unit, as is also the case for the first control unit.

The access control unit preferably still has a communication client for data exchange with the outside world, for example with a cloud. The communication client, for example, serves to transmit data with respect to the operation of the device to a digital log book in the cloud.

The communication client also preferably has no direct access to or no direct communication with the control unit(s) of the device, but can rather only communicate with it/them via the safety unit.

The communication of a device in accordance with the invention preferably always takes place via the access control unit. The access control unit is here preferably configured to communicate, preferably via a gateway, with the outside world and to receive control commands from, for example, a local network or from remote, for example via a cloud.

Alternatively or additionally, the communication of the device takes place via a display unit that preferably also acts as an input unit and is connected to the access control unit.

The display unit is here preferably part of the device and is, for example, designed as a screen, in particular a touchscreen, with the communication unit being connected to the display unit and being configured to determine and/or adapt contents displayed by the display unit.

Inputs, preferably with respect to the operation of the device, can still be actuated by means of the display unit; control commands can, for example, be input. Such inputs are communicated to the control unit(s) via the access control unit, in particular via the safety unit.

An IT infrastructure in accordance with the invention can generally be applied to any device A device in accordance with the invention is, however, preferably a device that is used as part of a blood treatment system.

The device can, for example, be a medical device, preferably a blood treatment device, in particular a dialysis machine.

The device can, however, also be a device for water preparation, preferably for preparing dialysis water, or for preparing a medical fluid. The device can, for example, be a component of a water preparation systems such as a reverse osmosis device, a filter device, or an associated sensor system. The device can also be a device for preparing a medical fluid, for example for preparing dialysis fluid from concentrates.

A further aspect of the invention relates to a use of a device in accordance with the invention in or with a dialysis system and/or to prepare a medical fluid and/or to prepare water, preferably to prepare dialysis water.

It is pointed out at this point that if an element is named in the singular within the framework of the present disclosure, the invention is not restricted thereto, but can also include the element in the plural, and vice versa.

In addition all the disclosed features, effects, and advantages of the invention can be isolated from one another or can be combined with one another.

Further advantages, features, and effects of the present invention result from the following description of an embodiment of the invention with reference to the Figures.

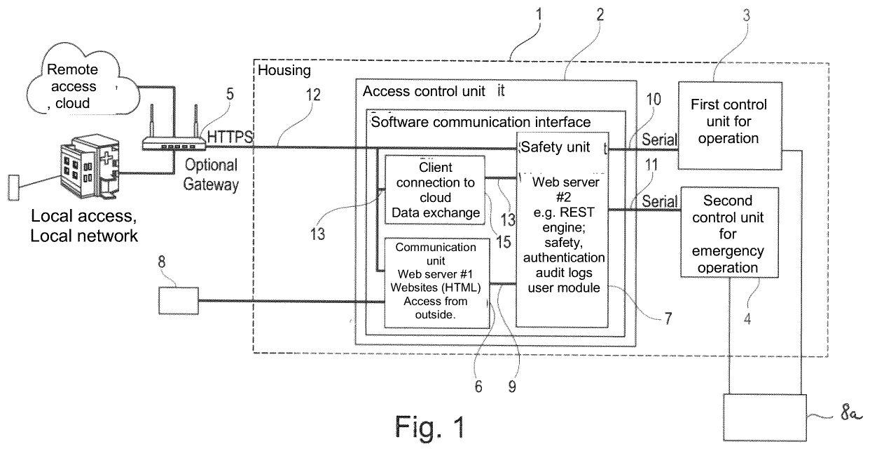

FIG. 1 schematically shows a device in accordance with the invention;

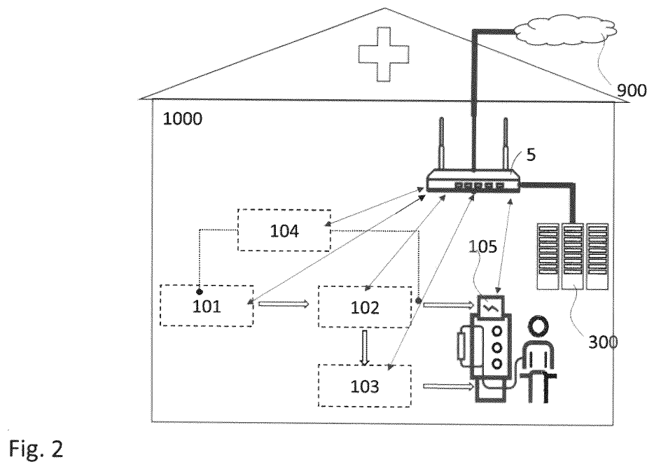

FIG. 2 schematically shows how a device in accordance with the invention or devices in accordance with the invention can be used, e.g. within a clinic.

As shown in FIG. 1, a device in accordance with the invention has a housing 1. An access control unit 2 and a first control unit 3 for the normal operation of the device and a second control unit 4 for emergency operation are respectively provided on separate circuit boards within the housing 1.

The access control unit 2 serves as a communication interface between the device and the outside world. For example, communication can take place from the outside via an optional gateway 5 with the device or initially with its access control unit 2; access to the device can here take place via remote access, for example from the cloud, or also via a local network; for example the local network of a clinic, that is connected to a blood treatment system.

The access control unit 2 has a communication unit 6 and a safety unit 7 that are formed in this embodiment as two separate and autonomous web servers (web server #1 and web server #2).

The communication device 6 is in communication with a display unit 8 of the device and generates a specific user interface thereon. Inputs that can be forwarded from the communication unit 6 to the safety unit 7 can also be actuated by means of the display unit 8, as is indicated by the communication flow 9. The display unit 8 is preferably a separate device such as a touch display, a tablet, or a laptop that is preferably wirelessly connected and/or connectable to the device.

The device moreover has a further display unit 8a by means of which inputs can also be actuated. The display unit 8a is in communication with the first control unit 3 and/or the second control unit and preferably enables a local or a direct control of the device 1, for example, in that control commands are locally directly input manually. The display unit 8a is preferably a part of the device 1 and is, for example, installed in and/or fixedly connected to the device.

The display units 8, 8a, that preferably also act as input units, can, for example, be fixedly installed at or in the device, for example in the form of a monitor or of a touchscreen, or it can be a respective end device such as a laptop or a tablet associated with the device 1.

In this embodiment, the communication unit 6 is as a web server capable of hosting web sites (e.g. html), but the communication unit 6 can also be configured to provide a different digital communication path which can be accessed from the outside via a network (internet, intranet, WAN, wireless LAN, LAN, etc.).

The safety unit 7 is in communication via connections 10 and 11, that are designed as a serial bus, for example, with the first and second control units 3 and 4. In the present embodiment, the safety unit 7 has a REST protocol (also a “REST engine”) as the communication protocol. This is, however, to be understood only by way of example and the safety unit 7 could also use any other communication protocol.

Only signals such as control commands coming from the outside that the safety unit 7 has checked as positive with respect to their safety, for example by checking user rolls, querying certificates, etc., are forwarded from the safety unit 7 to the control units 3 and 4.

As is indicated by the communication flow 12, signals coming from the outside such as control commands coming from clinic infrastructure software can reach the safety unit 7 directly. A device in accordance with the invention can thus always also be locally accessed to ensure safe operation even when a remote communication fails.

Alternatively or additionally, the safety unit 7, as is indicated by the communication flow 13, receives signals via a communication client 15 that is in data exchange with a cloud, for example. The communication client 15 is optional; in general, the access control unit 2 of a device in accordance with the invention can also only have the safety unit 7 and the communication unit 6. Alternatively, the access control unit 2 can be designed such that the communication unit 6 has a communication client for data exchange with a cloud, but no web server.

All the communication flows 12, 13, and 9 coming from the outside run, as is shown in FIG. 1, to the safety unit 7 that thus screens the control units 3 and 4 toward the outside. Only if a check of an incoming signal by the safety unit with respect to the technical safety assessment has given a positive result does the safety unit forward this signal to the control units 3 and 4.

The safety unit 7 thus forms a separation between a protected region that has the control units 3 and 4 and a region that is open to the outside world and that comprises the access control unit 2 and in particular the communication unit 6 and the communication client 15. The components for the control of the device that are relevant to the licensing can thus be reliably separated from other components that, for example, serve the adaptation or updating of user interfaces.

A device in accordance with the invention is thus characterized by a modular structure having at least two separate circuit boards or circuits in the same device/housing, wherein the one circuit board has a control unit that implements the hard control functions and the other circuit board has an access control unit that, in the form of the safety unit 7, implements the separation between the inside and the outside of the device in the sense of cyber security. At least two independent software instances that provide at least two different communication channels to external are provided on the latter circuit board.

A channel that is formed by the safety unit 7 enables a protocol-based direct passage by which information can be transmitted and control commands can be received. The second channel that is formed by the communication unit 6 and/or a communication client 15 enables a hosted and thus less direct communication—for example by a local web server or a link to a remote cloud.

A system 1000 is shown in FIG. 2 that comprises one or more devices in accordance with the invention and that is used in a clinic. Reference numeral 101 designates a device in accordance with the invention that is designed as a water pre-treatment device. Reference numeral 102 designates a device in accordance with the invention that is designed as a reverse osmosis system. Reference numeral 103 designates a device in accordance with the invention that is designed as a dialyzate concentrate preparation device or a dialyzate preparation device. Reference numeral 104 designates a device in accordance with the invention that is designed as a water treatment control room or as a water treatment sensor system device. Reference numeral 105 designates a device in accordance with the invention that is designed as a hemodialysis treatment device.

The system 1000 has at least one of the devices 101-105; it preferably has the devices 102 and 103. If the system should only be focused on the provision of liquid media, no device 105 is provided since this treatment machine does not provide any liquid media, but rather uses them. In this respect, one or more of the devices can be designed as a water pre-treatment device 101, a reverse osmosis system 102, a dialyzate concentrate preparation device, or a dialyzate preparation device 103, a water treatment control room or water treatment sensor system 104, a hemodialysis treatment device 105 in accordance with the invention. A system having such devices, with some being designed in accordance with the invention and some not being designed in accordance with the invention, is equally covered. The system can have one or more identical devices.

The devices 101, 102, 103, and 105 are connected to one another via the lines for liquid medium reproduced as arrows. The device 104 detects at least one parameter of the liquid medium at or in the device 101 and/or in the line between the reverse osmosis device 105 and the hemodialysis treatment device 105 by means of sensor lines or data connections.

The devices 101-105 can be in alternating data connection with a gateway 5. The gateway 5 is, however, completely optional in the context of the invention. One or more of the devices of water pre-treatment device 101, reverse osmosis system 102, dialyzate concentrate preparation device or dialyzate preparation device 103, and water treatment control room or water treatment sensor system device 104 can in particular also be connected without any gateway 5 directly to the network of a clinic, of a hospital, of a treatment center, or to the internet or a cloud. The gateway 5 is connected to a local computing center 300 or to the local IT structure of the clinic and is equally in communication with the cloud 900.

1. A device for preparing a medical fluid and/or for monitoring the preparation of a medical fluid comprising:
a housing;
a first control unit that controls the device in operation; and
an access control unit that forms at least one interface between the device and the outside world,
wherein the first control unit is configured also to control the device on a failure of the access control unit, and the control unit and the access control unit are each configured on a separate circuit board arranged in the housing; and
the access control unit furthermore has
a communication unit for communication with the outside world; and
a safety device that is formed separately from the communication unit and that is configured to check every communication between the first control unit and the outside world via the interface and only to permit it after a determined technical safety assessment.

2. The device in accordance with claim 1, wherein the device has at least two separate interfaces between the access control unit and the outside world; and wherein the safety unit is configured to check every communication of the first control unit with the outside world over any interface of the device with the outside world and only to permit it after a determined technical safety assessment.

3. The device in accordance with claim 2, wherein the safety unit is in communication with the outside world over at least two separate communication paths, with a first communication path coming from the outside first leading to the communication unit and thereupon to the safety unit and a second communication path coming from the outside leading directly to the safety unit and the safety unit being configured to check any communication of the first control unit over the first and second communication paths and only to permit it after a determined technical safety assessment.

4. The device in accordance with claim 1, wherein the communication unit and the safety unit are implemented as two separate web servers and/or as two separate autonomous parts of a communication module.

5. The device in accordance with claim 1, furthermore having a second control unit that controls the device in emergency operation, with the second control unit being configured on a separate, third board arranged in the housing and the safety unit furthermore being configured to check every communication of the second control unit with the outside world.

6. The device in accordance with claim 1, wherein the safety unit forms a technical safety separation between a first region of the device and a second region of the device, with the first region having the communication unit and the second region having the first, and optionally the second, control unit.

7. The device in accordance with claim 1, wherein the communication unit is configured to provide a communication path for network communication.

8. The device in accordance with claim 1, wherein at least information from the first control unit can be transferred to the outside by means of the first communication path and information from the first control unit can be transferred to the outside by means of the second communication path and/or information can be transferred from the outside to the control unit and/or access to the first safety unit can take place from the outside if the safety unit has determined a technical safety assessment.

9. The device in accordance with claim 1, furthermore having at least one display unit that is part of the device,
wherein the display unit is connected to the first control unit.

10. The device in accordance with claim 9, wherein inputs with respect to the operation of the device are capable of being actuated by means of the display unit or of an additional input device that is part of the device.

11. The device in accordance with claim 1, wherein the device is a medical device and/or a device for providing medical water or medical solutions.

12. The device in accordance with claim 6, wherein the second region of the device is configured as a medical device and the first region of the device is not configured as a medical device.

13. The device in accordance with claim 11, wherein the device is a blood treatment device.

14. The device in accordance with claim 11, wherein the device is a device for water pre-treatment, a reverse osmosis system, a concentrate mixing system, or a technical water control system.

15. The device in accordance with claim 1, wherein the device is further configured to detect a parameter of the medical fluid, which is optionally pure medical water, by means of at least one sensor, with the sensor either being a component of the device or being connected to the device via a line.

16. A method for preparing a medical fluid and/or for water preparation, said method comprising utilizing the device of claim 1.

17. The method of claim 16, wherein the method is conducted at a clinic or a dialysis center, and the medical fluid is a liquid medium for dialysis.

18. The device in accordance with claim 5, furthermore having at least one display unit that is part of the device and that is configured as a screen, wherein the display unit is connected to the first control unit and also to the second control unit.

19. The device in accordance with claim 1, furthermore having at least one display unit that is part of the device and that is configured as a screen, wherein the display unit is connected to the first control unit.

20. The device in accordance with claim 1, wherein the communication unit is configured to provide a communication path for network communication such that the communication unit hosts a web site by means of a web server and/or hosts a cloud service and/or provides a connection to a cloud server.
